(19) United States
(12) Patent Application Publication
(10) Pub. No.: US 2021/0236842 A1
(43) Pub. Date: Aug. 5, 2021

(54) LIGHT IRRADIATION TREATMENT INSTRUMENT AND OPERATION METHOD FOR LIGHT IRRADIATION TREATMENT INSTRUMENT

(71) Applicant: OLYMPUS CORPORATION, Tokyo (JP)

(72) Inventors: Koji MATSUMOTO, Tokyo (JP); Toshiaki SUZUKI, Tokyo (JP); Takeshi YAMAZAKI, Tokyo (JP); Masahiro YOSHINO, Tokyo (JP)

(73) Assignee: OLYMPUS CORPORATION, Tokyo (JP)

(21) Appl. No.: 17/235,006

(22) Filed: Apr. 20, 2021

Related U.S. Application Data
(63) Continuation of application No. PCT/JP2018/044763, filed on Dec. 5, 2018.

(51) Int. Cl. A61N 5/06

(52) U.S. Cl. CPC A61N 5/0603; A61B 2018/00285; A61N 2005/063; A61N 2005/061

(57) ABSTRACT

A light radiating unit provided at a distal end portion of an optical fiber includes a first light radiating unit having a predetermined length in a longitudinal direction and capable of radiating therapeutic light having first intensity and a second light radiating unit connected consecutively to the first light radiating unit in the longitudinal direction and configured to radiate therapeutic light having second intensity lower than the first intensity. Consequently, even when a bladder, which is a hollow organ, and a urethra, which is a conduit, are irradiated with the therapeutic light at a time, intensity of the therapeutic light with which surfaces (inner wall surfaces) of respective parts are irradiated is equalized.

### CROSS REFERENCE TO RELATED APPLICATION

This application is a continuation application of PCT/JP2018/044763 filed on Dec. 5, 2018, the entire contents of which are incorporated herein by this reference.

### BACKGROUND OF THE INVENTION

### 1. Field of the Invention

The present invention relates to a light irradiation treatment instrument that guides optical energy into a body cavity as therapeutic light and irradiates a lesion part or the like with the therapeutic light and an operation method for the light irradiation treatment instrument.

### 2. Description of the Related Art

In recent years, in a medical field, PDT (photodynamic therapy), PIT (photoimmuno therapy), and the like have been studied as methods of treatment for killing cancer cells using optical energy.

For example, as disclosed in Japanese Patent No. 6127045, PIT includes a step of bringing a therapeutically effective amount of antibody molecules into contact with a cell having cell surface protein, a step of specifically combining antibodies with cell surface protein such as a tumor-specific antigen on a surface of a tumor cell, and a step of irradiating a cell with optical energy such as a laser beam to break up a cell membrane.

As a technique for irradiating, with PDT treatment or the like, a hollow organ such as a bladder with light, for example, Japanese Patent Application Laid-Open Publication No. S59-95065 discloses a technique for, on an inside of a hollow organ such as a bladder, expanding a balloon provided at a distal end of a catheter with a scattering medium and uniformly irradiating, with the scattering medium, a hollow organ inner wall surface with light from a light guide (a light irradiation treatment instrument).

A lesion in a bladder tends to often occur in a connecting portion to a urethra where urine easily accumulates. Therefore, when the bladder is treated by PDT or PIT, it is desirable to irradiate not only an inside of the bladder but also a urethra portion (a proximal urethra portion) with optical energy.

### SUMMARY OF THE INVENTION

A light irradiation treatment instrument according to an aspect of the present invention includes: an insertion member insertable into an inside of a living body; a positioning unit disposed between a first end and a second end of the insertion member and capable of positioning the insertion member with respect to the living body; and a light radiating unit disposed between the first end and the second end and configured to radiate therapeutic light toward an outer side, intensity of the therapeutic light being different between a side closer to the first end than the positioning unit and a side closer to the second end than the positioning unit.

A light irradiation treatment instrument according to another aspect of the present invention includes: a catheter main body having translucency and being insertable into a conduit and an inside of a hollow organ communicating with the conduit; a balloon having translucency and being provided in the catheter main body and configured to expand to thereby position the catheter main body with respect to the conduit and the hollow organ; and an optical fiber having a predetermined length in a longitudinal direction, a first light radiating unit capable of radiating therapeutic light having first intensity and a second light radiating unit connected consecutively to the first light radiating unit in the longitudinal direction and capable of radiating the therapeutic light having second intensity different from the first intensity being formed on a distal end side, the optical fiber guiding the therapeutic light to the first light radiating unit and the second light radiating unit. When the optical fiber is inserted, the catheter main body positioned by the balloon positions one of the first light radiating unit and the second light radiating unit on an inside of the hollow organ and positions another one of the first light radiating unit and the second light radiating unit on an inside of the conduit and a joint portion of the conduit and the hollow organ.

An operation method for a light irradiation treatment instrument according to an aspect of the present invention includes: inserting a catheter main body into a conduit and an inside of a hollow organ communicating with the conduit; positioning the catheter main body with respect to the conduit and the hollow organ; inserting an optical fiber into an inside of the catheter main body, the optical fiber having a predetermined length in a longitudinal direction, a first light radiating unit capable of radiating therapeutic light having first intensity and a second light radiating unit connected consecutively to the first light radiating unit in the longitudinal direction and capable of radiating therapeutic light having second intensity different from the first intensity being formed on a distal end side; and, with respect to the catheter main body, positioning one of the first light radiating unit and the second light radiating unit on an inside of the hollow organ and positioning another one of the first light radiating unit and the second light radiating unit on an inside of the conduit and a joint portion of the conduit and the hollow organ.

### BRIEF DESCRIPTION OF THE DRAWINGS

FIG. 1 is a schematic configuration diagram of an optical treatment system according to a first embodiment of the present invention;

FIG. 2 is an explanatory diagram of a case in which light irradiation treatment into a bladder is performed using the optical treatment system according to the first embodiment of the present invention;

FIG. 2A is a main part sectional view of a light radiating unit according to the first embodiment of the present invention;

FIG. 2B is a main part sectional view of the light radiating unit according to the first embodiment of the present invention;

FIG. 2C is a main part sectional view of the light radiating unit according to the first embodiment of the present invention;

FIG. 3 is an explanatory diagram of a case in which light irradiation treatment into a bladder is performed using an optical treatment system according to a first modification of the first embodiment of the present invention;

FIG. 3A is a main part sectional view of a light radiating unit according to the first modification of the first embodiment of the present invention;

FIG. 4 is an explanatory diagram of a case in which light irradiation treatment into a bladder is performed using an optical treatment system according to a second modification of the first embodiment of the present invention;

FIG. 5 is an explanatory diagram of a case in which light irradiation treatment into a bladder is performed using an optical treatment system according to a third modification of the first embodiment of the present invention;

FIG. 5A is a main part sectional view of a light radiating unit according to the third modification of the first embodiment of the present invention;

FIG. 6 is a main part sectional view of a core in the light radiating unit according to the third modification of the first embodiment of the present invention;

FIG. 7 is an explanatory diagram of a case in which light irradiation treatment into a bladder is performed using an optical treatment system according to a fourth modification of the first embodiment of the present invention;

FIG. 8 is a main part sectional view of a light radiating unit according to the fourth modification of the first embodiment of the present invention;

FIG. 9 is an explanatory diagram of a case in which light irradiation treatment into a bladder is performed using an optical treatment system according to a fifth modification of the first embodiment of the present invention;

FIG. 10 is a schematic configuration diagram of an optical treatment system according to a sixth modification of the first embodiment of the present invention;

FIG. 11 is an explanatory diagram of a case in which light irradiation treatment into a bladder is performed using the optical treatment system according to the sixth modification of the first embodiment of the present invention;

FIG. 12 is an explanatory diagram of a case in which light irradiation treatment into a bladder is performed using an optical treatment system according to a second embodiment of the present invention;

FIG. 13 is an explanatory diagram of a case in which light irradiation treatment into a bladder is performed using an optical treatment system according to a first modification of the second embodiment of the present invention;

FIG. 14 is a main part sectional view of an optical fiber around which a second optical fiber is disposed according to the first modification of the second embodiment of the present invention;

FIG. 15 is an explanatory diagram of a case in which light irradiation treatment into a bladder is performed using an optical treatment system according to a second modification of the second embodiment of the present invention;

FIG. 16 is a main part sectional view of a light radiating unit according to the second modification of the second embodiment of the present invention;

FIG. 17 is an explanatory diagram of a case in which light irradiation treatment into a bladder is performed using an optical treatment system according to a third modification of the second embodiment of the present invention;

FIG. 18 is a main part sectional view of a light radiating unit according to the third modification of the second embodiment of the present invention; and

FIG. 19 is a flowchart showing a therapeutic light radiation control routine according to a fourth modification of the second embodiment of the present invention.

### DETAILED DESCRIPTION OF THE PREFERRED EMBODIMENTS

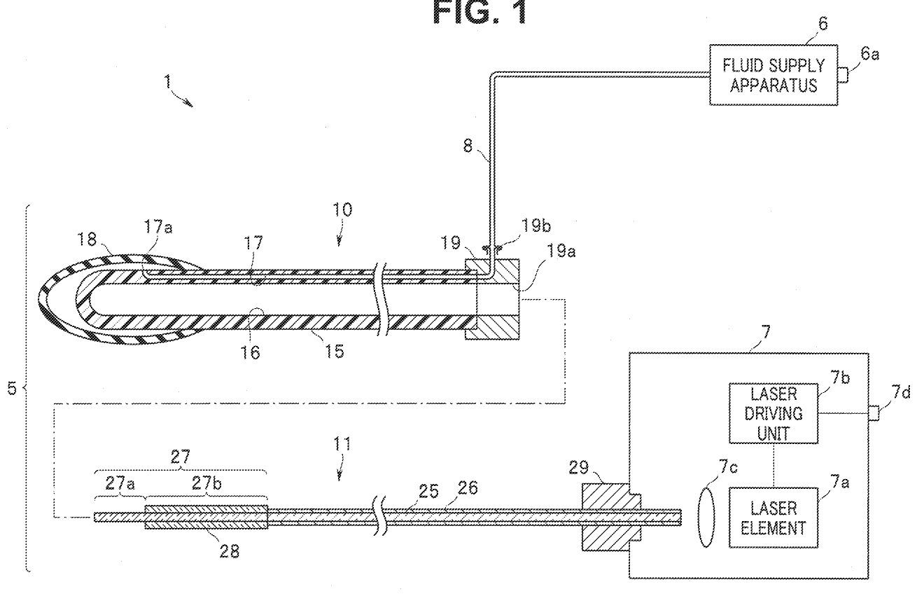

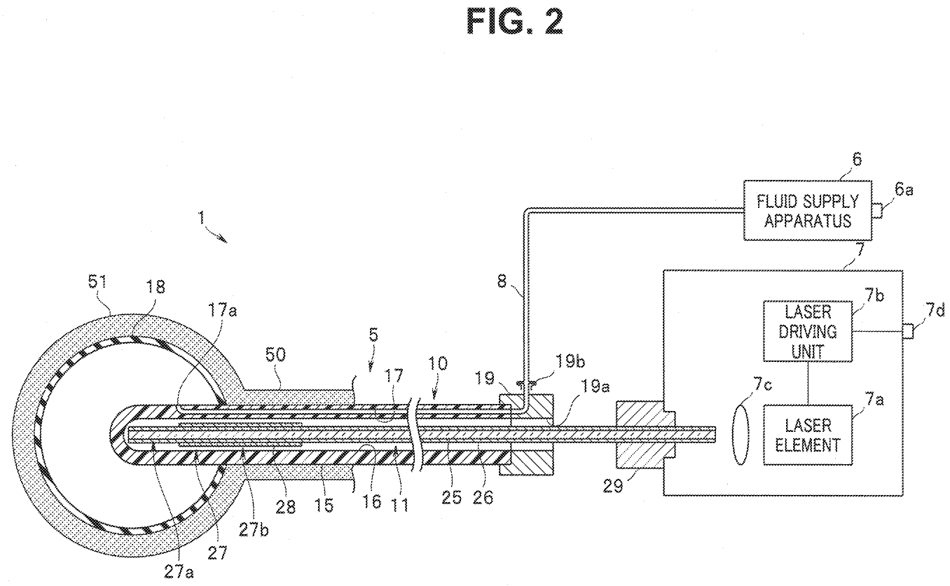

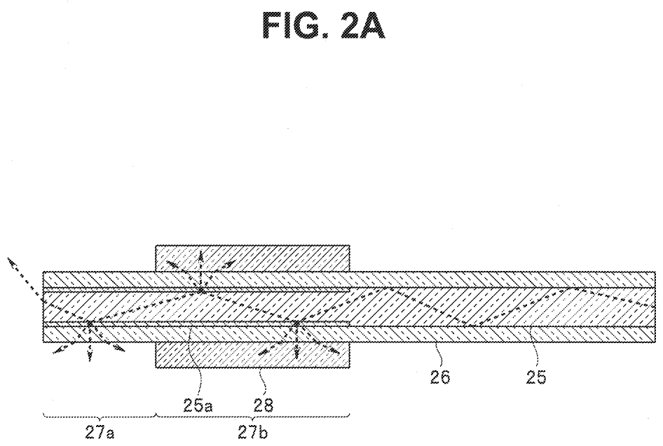

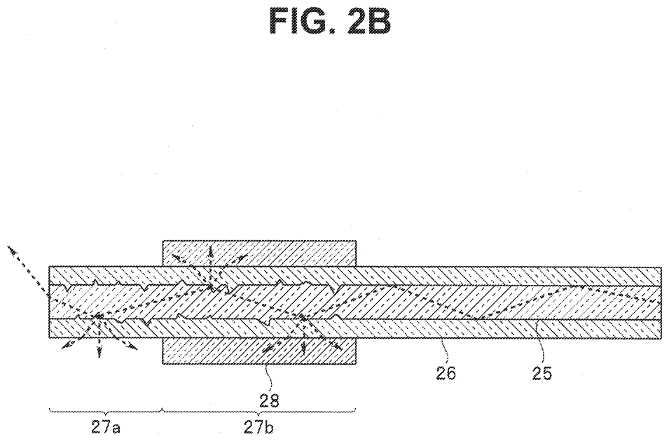

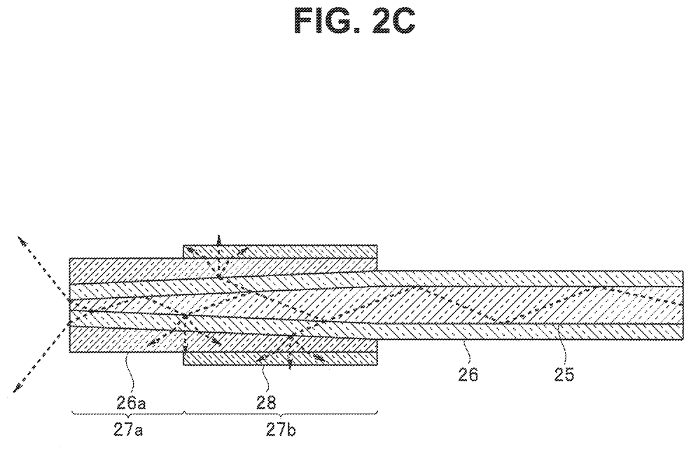

Embodiments of the present invention are explained below with reference to the drawings. FIGS. 1 and 2 relate to a first embodiment of the present invention. FIG. 1 is a schematic configuration diagram of an optical treatment system. FIG. 2 is an explanatory diagram of a case in which light irradiation treatment into a bladder is performed using the optical treatment system.

A laser treatment system 1 functioning as the optical treatment system shown in FIG. 1 is, for example, a system for irradiating a diseased part in a living body with, as therapeutic light, a high-power laser beam (a near infrared laser beam) matching treatment such as PIT (photoimmuno therapy).

The laser treatment system 1 includes a light irradiation treatment instrument 5 inserted into a living body (for example, a bladder) to irradiate a diseased part of the living body with therapeutic light, a fluid supply apparatus 6 for supplying fluid to the light irradiation treatment instrument 5, and a light source apparatus 7 for supplying therapeutic light such as a laser beam to the light irradiation treatment instrument 5.

The light irradiation treatment instrument 5 includes a catheter 10 functioning as an insertion member insertable into an inside of a living body (for example, into a urethra 50 and a bladder 51) and an optical fiber 11 inserted into the inside of the living body via the catheter 10 to irradiate a diseased part of the living body with therapeutic light.

The catheter 10 in the present embodiment is, for example, a disposable catheter for urinary organs, both ends in a longitudinal direction of which are set as a first end and a second end. The catheter 10 includes an elongated catheter main body 15 having flexibility and having a transparent characteristic with respect to therapeutic light emitted from the optical fiber 11.

The catheter main body 15 is configured by, for example, a multi-lumen tube including a first conduit 16 into which the optical fiber 11 is insertable and a second conduit 17 capable of allowing fluid such as air to flow therethrough.

At a distal end portion of the catheter main body 15, a distal end of the first conduit 16 is closed and a distal end of the second conduit 17 is opened to a side.

A balloon 18 is provided at the distal end portion of the catheter main body 15. The balloon 18 is formed in a bag shape and is disposed at the distal end portion of the catheter main body 15 in a position where the balloon 18 covers a distal end opening section 17a of the second conduit 17.

An opened end portion of the balloon 18 is liquid-tightly fixed to an outer circumferential surface of the catheter main body 15 by not-shown bobbin bonding or the like.

A closed space communicating with the distal end opening section 17a of the second conduit 17 is formed at the distal end portion of the catheter main body 15 by the balloon 18 fixed in this way.

The balloon 18 is formed by an elastic body such as rubber having a transparent characteristic with respect to the therapeutic light emitted from the optical fiber 11.

A pipe sleeve 19 is connected to a proximal end side of the catheter main body 15. In the pipe sleeve 19, a treatment instrument insertion port 19a communicating with the first conduit 16 and a connector section 19b communicating with the second conduit 17 are provided.

The fluid supply apparatus 6 of a pump type is connected to the connector section 19b, for example, via a fluid conduit 8. A pump driving switch 6a is provided in the fluid supply apparatus 6.

When a distal end portion of the catheter 10 is inserted into an inside of the bladder 51 through the urethra 50 and the pump driving switch 6a is turned on, the fluid supply apparatus 6 compresses the fluid such as air and supplies the fluid into the second conduit 17 of the catheter main body 15. Consequently, the fluid supply apparatus 6 is capable of expanding the balloon 18 (see FIG. 2).

The fluid supply apparatus 6 in the present embodiment is set to supply, to the balloon 18, the fluid in a constant amount set by an experiment, a simulation, or the like in advance.

More specifically, an amount of the fluid supplied from the fluid supply apparatus 6 to the balloon 18 is set to, for example, an amount appropriate for the balloon 18 to extend the bladder 51 with expansion and stretching wrinkles of an inner wall of the bladder 51.

By expanding the balloon 18 with the preset constant amount of the fluid in this way, it is possible to always extend the inside of the bladder 51 to a constant volume with the balloon 18.

The expanded balloon 18 comes into contact with the inner wall of the bladder 51 over substantially an entire surface, whereby a fixed portion of the balloon 18 to the catheter main body 15 is pressed against a vicinity of a joint portion of the bladder 51 and the urethra 50. Consequently, the catheter 10 is positioned with respect to the bladder 51 or the like based on the fixed portion of the balloon 18. In other words, the catheter 10 is positioned such that the distal end portion of the catheter main body 15 is always disposed on the inside of the bladder 51 by a preset amount. In this way, the balloon 18 realizes a function of a positioning member for positioning the catheter 10. The fixed portion of the balloon 18 realizes a function of a positioning section.

The optical fiber 11 includes a core 25 and a clad 26 covering an outer circumference of the core 25. The core 25 and the clad 26 in the present embodiment is formed by, for example, quartz. Impurities for setting a refractive index of the clad 26 lower than a refractive index of the core 25 are added to the clad 26. Light is totally reflected on a boundary surface between the core 25 and the clad 26 to be guided. Note that a material configuring the core 25 and the clad 26 is not limited to the quartz. For example, resin having translucency can also be adopted.

At a distal end portion of the optical fiber 11, a light radiating unit 27 for radiating, to an irradiation target in a body cavity, therapeutic light guided from a proximal end side to a distal end side of the core 25 is provided.

The light radiating unit 27 in the present embodiment is configured by a substantially linear member having a predetermined length in the longitudinal direction, for example, in order to irradiate a region from the bladder to a urethra portion, which is an irradiation target, with the therapeutic light.

For example, as shown in FIG. 2A, in the light radiating unit 27, a diffusing agent 25a is added to a part of the core 25 or a boundary part between the core 25 and the clad 26 to thereby diffuse a part of the guided light in a side surface direction. Alternatively, for example, as shown in FIG. 2B, in the light radiating unit 27, fine unevenness machining is applied to a surface of a boundary between the core 25 and the clad 26 to thereby diffuse a part of the guided light in the side surface direction. Alternatively, for example, as shown in FIG. 2C, the light radiating unit 27 is imparted with, in a direction in which the core 25 and the clad 26 guide light, a gradient to break the total reflection condition and leak a part of the guided light in the side surface direction. Further, a part of the guided light is diffused in the side surface direction by adding a diffusing agent 26a to the clad 26 or a clad surface.

A light attenuating unit 28 made of a covering member having low transmittance is provided in an outer circumference on a proximal end side of the light radiating unit 27. Consequently, a first light radiating unit 27a and a second light radiating unit 27b having different intensities of light per unit area from each other are set.

In other words, in the light radiating unit 27, a region on a distal end side exposed from the light attenuating unit 28 is set as the first light radiating unit 27a for radiating therapeutic light to mainly a region in the bladder 51 at a large distance from the light radiating unit 27 (in an irradiation target, mainly a region of the irradiation target within a first distance range in which a shortest distance from the light radiating unit 27 is equal to or larger than a predetermined distance).

On the other hand, in the light radiating unit 27, a region on the proximal end side covered by the light attenuating unit 28 is set as the second light radiating unit 27b having the intensity of light per unit area smaller than the intensity of light per unit area of the first light radiating unit 27a. The second light radiating unit 27b is a light radiating unit for radiating therapeutic light to mainly an inside of the urethra 50 and the vicinity of the joint portion to the urethra 50 in the bladder 51 at a small distance from the light radiating unit 27 (in the irradiation target, a region of the irradiation target within a second distance range that is smaller than the first distance range and in which the shortest distance from the light radiating unit 27 is smaller than the predetermined distance).

The optical fiber 11 is inserted into the first conduit 16 and positioned with respect to the catheter 10, whereby the first and second light radiating units 27a and 27b are positioned with respect to the bladder 51 and the urethra 50.

More specifically, as explained above, the catheter 10 is positioned with respect to the bladder 51 or the like by the expanded balloon 18.

For example, the optical fiber 11 is inserted to a position where the optical fiber 11 is hit against the distal end of the first conduit 16. The optical fiber 11 is positioned with respect to the catheter 10. Note that the positioning of the optical fiber 11 with respect to the catheter 10 can also be realized by, for example, providing a mark for positioning in an outer circumference of the optical fiber 11 and positioning the mark in the pipe sleeve 19 or the like of the catheter 10.

In this way, the optical fiber 11 is positioned with respect to the catheter positioned with respect to the bladder 51 or the like. Consequently, the first and second light radiating units 27a and 27b formed in the optical fiber 11 are positioned with respect to the bladder 51 and the urethra 50 via the catheter 10.

In other words, for example, as shown in FIG. 2, the first light radiating unit 27a is positioned in a region closer to a center in the bladder 51. The second light radiating unit 27b is positioned in a region from an inside of the urethra 50 to the vicinity of the joint portion to the urethra 50 in the bladder 51.

When therapeutic light is supplied from the light source apparatus 7 explained below to the proximal end side of the optical fiber 11, the first and second light radiating units 27a and 27b are capable of irradiating surfaces (inner wall surfaces) of respective parts of the bladder 51 and the urethra 50 with therapeutic light having uniform intensity.

In other words, since the balloon 18 in the present embodiment extends the bladder 51 to the constant volume with the constant amount of the fluid, a distance from the first light radiating unit 27a disposed on an inside of the balloon 18 to the inner wall of the bladder 51 is substantially unconditionally decided. In general, since the outer circumference of the catheter 10 inserted into the urethra 50 comes into contact with the inner wall of the urethra 50, a distance from the second light radiating unit 27b to the inner wall of the urethra 50 or the like is also substantially unconditionally decided. By optimizing the intensity of the therapeutic light attenuated by the light attenuating unit 28 in the second light radiating unit 27b considering a relation among these distances, it is possible to equalize the intensity of the therapeutic light with which the surfaces (the inner wall surfaces) of the respective parts of the bladder 51 and the urethra 50 are irradiated.

The proximal end side of the optical fiber 11 configured in this way is detachably connected to the light source apparatus 7 via an optical connector 29. In other words, the proximal end side of the optical fiber 11 is fixed to the optical connector 29 in a state in which the proximal end side of the optical fiber 11 is pierced through the optical connector 29. Consequently, when the optical connector 29 is connected to the light source apparatus 7, the optical connector 29 is capable of positioning a proximal end of the optical fiber 11 in a predetermined position on an inside of the light source apparatus 7.

The light source apparatus 7 includes a laser element 7a and a laser driving unit 7b.

The laser element 7a is configured by, for example, a laser diode capable of emitting an infrared laser beam. The laser element 7a is disposed in a position opposed to, via a lens 7c, the proximal end of the optical fiber 11 positioned on the inside of the light source apparatus 7. Consequently, the laser element 7a is capable of making a laser beam functioning as therapeutic light incident on the optical fiber 11.

When a light source driving switch 7d is turned on, the laser driving unit 7b controls to drive the laser element 7a for a preset setting time. Consequently, the respective parts of the bladder 51 and the urethra 50 faced to the light radiating unit 27 are irradiated with the therapeutic light at uniform intensity. Uniform radiation energy is imparted to the respective parts of the bladder 51 and the urethra 50.

According to such an embodiment, the light radiating unit 27 provided at the distal end portion of the optical fiber 11 includes the first light radiating unit 27a having a predetermined length in the longitudinal direction and capable of radiating therapeutic light having first intensity and the second light radiating unit 27b that is connected consecutively to the first light radiating unit 27a in the longitudinal direction and radiates therapeutic light having second intensity lower than the first intensity. Consequently, even when the bladder 51, which is a hollow organ, and the urethra 50, which is a conduit, are irradiated with therapeutic light at a time, it is possible to equalize intensity of the therapeutic light with which the surfaces (the inner wall surfaces) of the respective parts are irradiated.

In other words, the intensity of the therapeutic light radiated by the first light radiating unit 27a and the intensity of the therapeutic light radiated by the second light radiating unit 27b are adjusted according to a difference between the distance from the light radiating unit 27 to the inner wall surface of the bladder 51 and the distance from the light radiating unit 27 to the inner wall surface of the urethra 50 or the like. Consequently, even when the bladder 51, which is the hollow organ, and the urethra 50, which is the conduit, are irradiated with the therapeutic light at a time, it is possible equalize the intensity of the therapeutic light with which the surfaces (the inner wall surfaces) of the respective parts are irradiated. Therefore, it is possible to impart more uniform energy in the same irradiation time to the respective parts of the bladder 51, which is the hollow organ, and the urethra 50, which is the conduit.

In this case, by expanding the balloon 18 with a preset constant amount of the fluid, it is possible to always extend the inside of the bladder 51, which is the hollow organ, to the constant volume with the balloon 18 and it is possible to unconditionally decide the distance from the first light radiating unit 27a to the inner wall surface of the bladder 51 at the time when the therapeutic light is irradiated.

When the balloon 18 is expanded in a hollow organ such as the bladder 51, the fixed portion of the balloon 18 to the catheter main body 15 is brought into contact with the vicinity of the joint portion of the bladder 51 and the urethra 50. The catheter 10 (the catheter main body 15) is positioned in the bladder 51 and the urethra 50 based on the contact of the fixed portion of the balloon 18 and the vicinity of the joint portion. By positioning the optical fiber 11 with respect to the catheter 10 positioned in this way, it is possible to accurately position the first light radiating unit 27a and the second light radiating unit 27b with respect to the bladder 51 and the urethra 50.

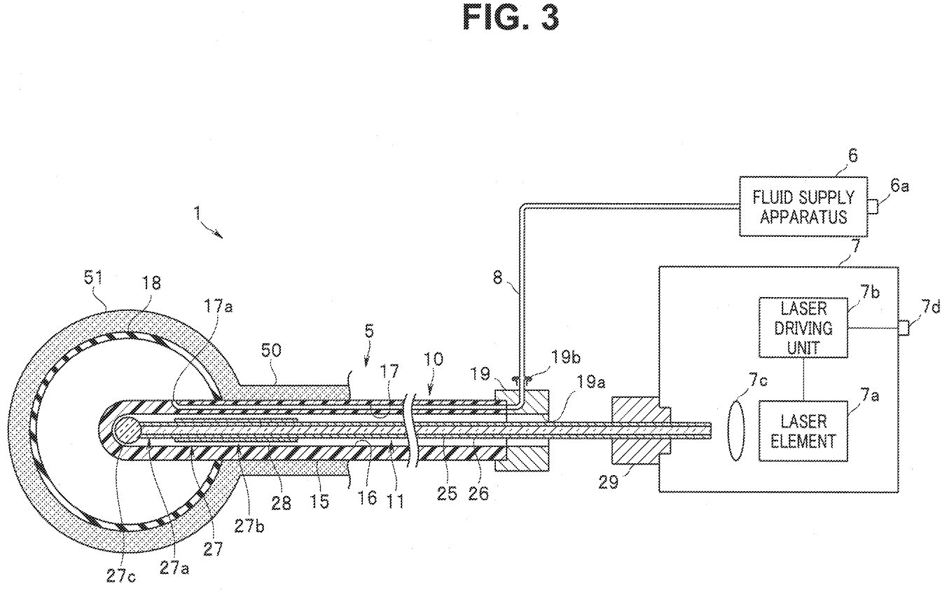

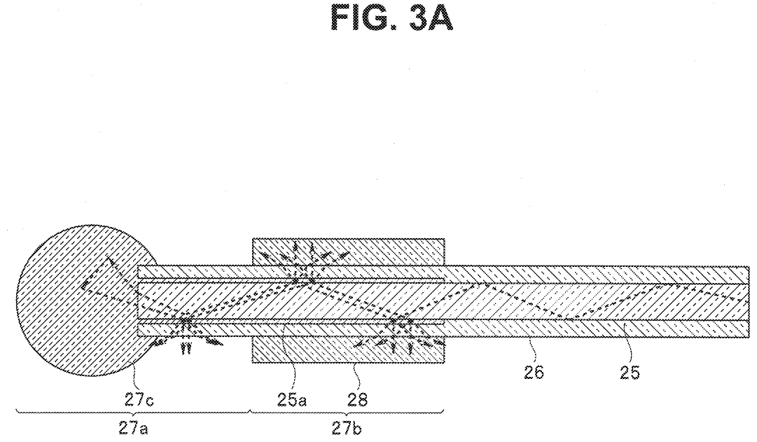

In the embodiment explained above, for example, as shown in FIGS. 3 and 3A, it is also possible to form a spherical portion 27c having a predetermined length in the longitudinal direction at a distal end of the first light radiating unit 27a. The spherical portion 27c diffuses light emitted from the core 25 with a diffusing agent included in the spherical portion 27c. The inside of the bladder 51 is irradiated at uniform intensity with the light diffused by the diffusing agent. A part of the light is guided to the core 25 and used as light irradiated from the light radiating unit 27a and the light radiating unit 27b.

With such a configuration, if the spherical portion 27c is set to be positioned in the center of the extended bladder 51 during the expansion of the balloon 18, it is possible to irradiate the inside of the bladder 51 with the therapeutic light at more uniform intensity.

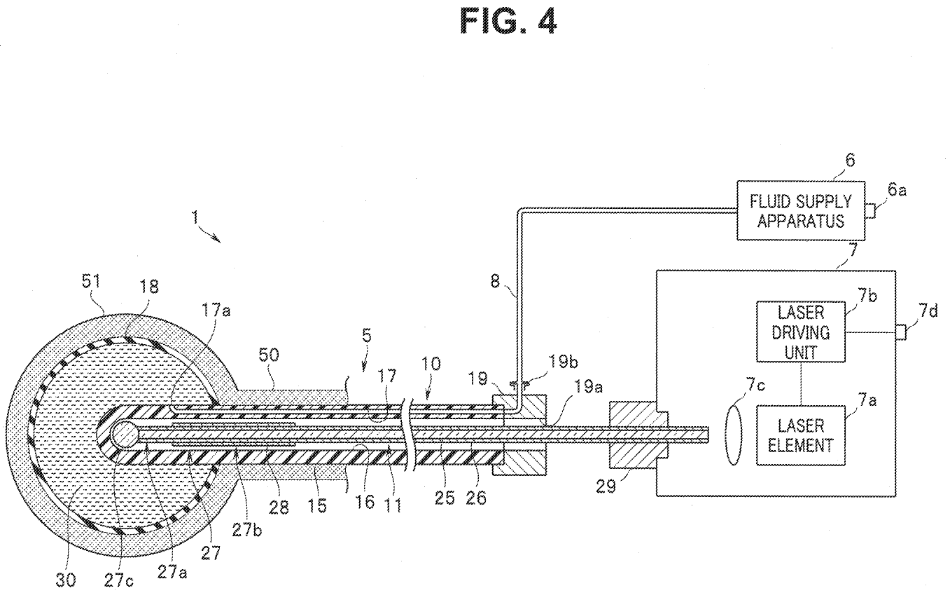

For example, as shown in FIG. 4, as the fluid supplied to the inside of the balloon 18, it is also possible to use liquid 30 such as a physiological saline or a scattering substance instead of gas such as air. In other words, it is possible to cause the balloon 18 to function as a liquid holding unit that holds light-transmittable liquid around the first light radiating unit.

For example, by using the scattering substance as the liquid 30, it is possible to efficiently scatter the therapeutic light radiated from the first light radiating unit 27a. It is possible to radiate the therapeutic light having more uniform intensity to the respective parts in the bladder 51.

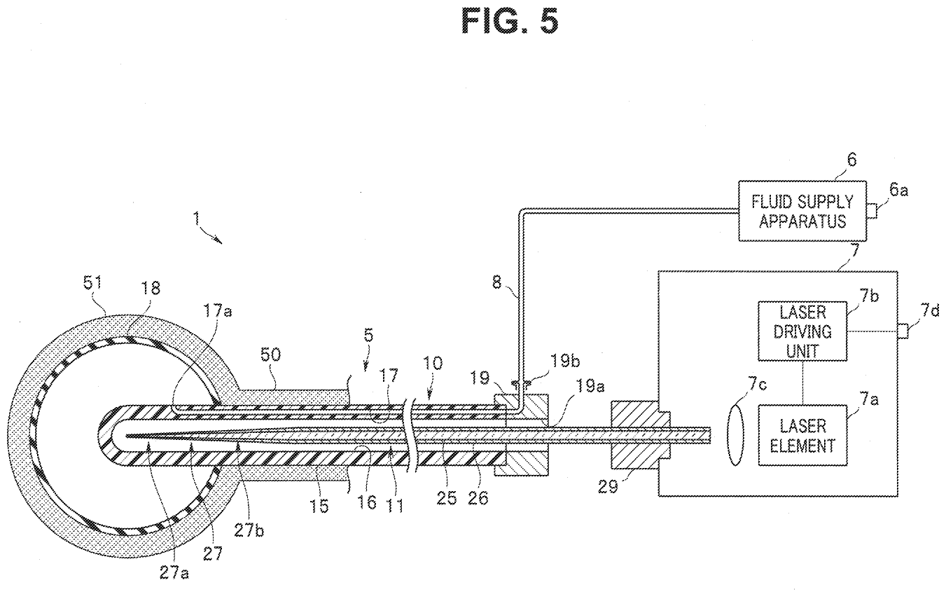

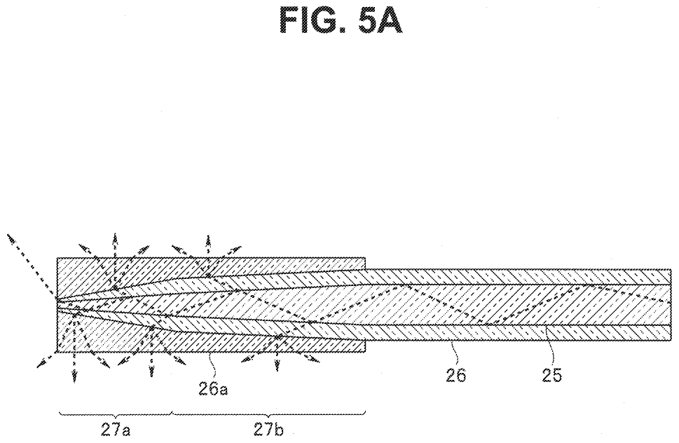

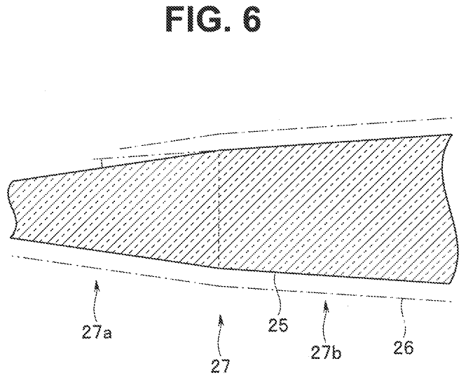

For example, as shown in FIGS. 5, 5A, and 6, the first and second light radiating units 27a and 27b have different gradients from each other in a direction in which the core 25 and the clad 26 guide light. The first and second light radiating units 27a and 27b break the total reflection condition with these gradients to thereby change intensity of leaking a part of the guided light in the side surface direction. The leaked light is diffused in the side surface direction by adding the diffusing agent 26a to the clad 26.

In other words, in the present modification, the first and second light radiating units 27a and 27b are realized by forming an outer circumferential surface of the core 25 with two-stage conical surfaces. More specifically, as shown in FIG. 5, an outer circumferential surface of the first light radiating unit 27a is formed by a conical surface having a preset first inclination angle. An outer circumferential surface of the second light radiating unit 27b is formed by a conical surface having a preset second inclination angle smaller than the first inclination angle. In FIG. 5A, a shape in which the clad 26 forms two-stage conical surfaces like the outer circumferential surface of the core 25 is shown. However, concerning the shape of the clad 26, the two-stage conical surfaces do not always need to be formed. The clad 26 may form a one-stage conical surface or may not form a conical surface and may form a columnar shape without a stage.

These first and second inclination angles are set according to intensities of therapeutic light requested for the first and second light radiating units 27a and 27b and are set based on an experiment or a simulation in advance.

The outer circumferential surface is formed by the conical surface in this way. Consequently, the first and second light radiating units 27a and 27b are capable of radiating, at intensities corresponding to the inclination angles, the therapeutic light guided to the distal end side while totally reflecting in the core 25 of the optical fiber 11.

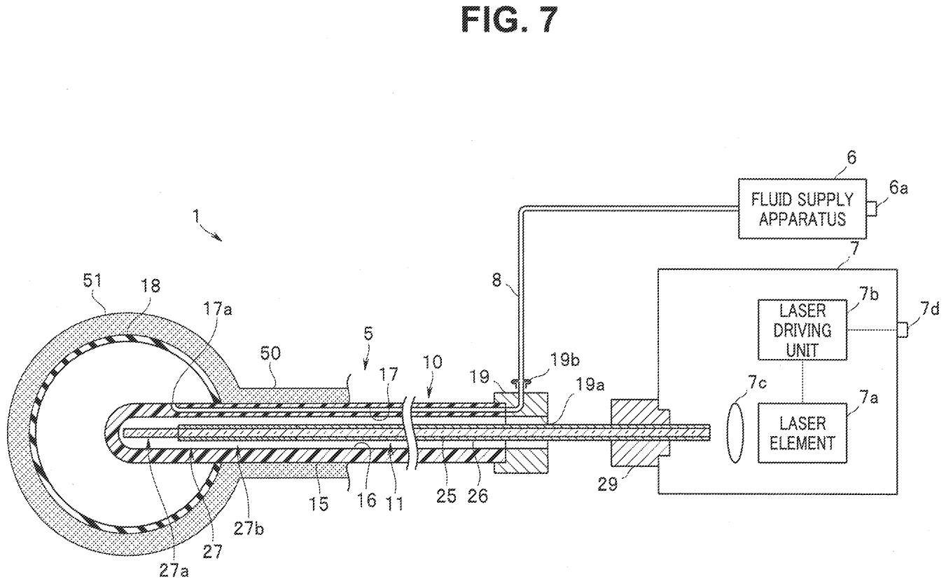

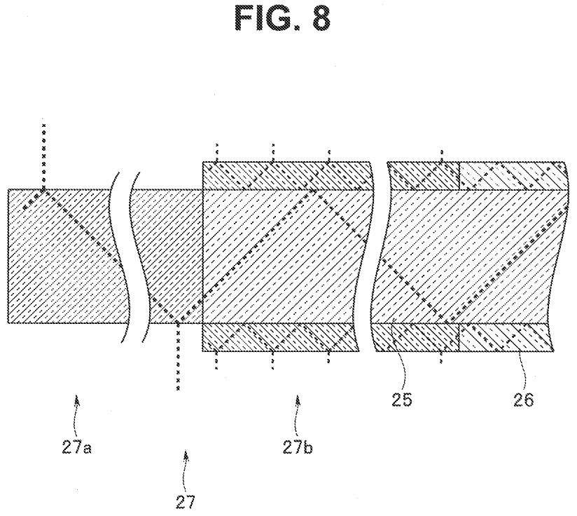

For example, as shown in FIGS. 7 and 8, it is also possible to form the second light radiating unit 27b using the clad 26.

In other words, although a laser beam is made incident on only a core and guided, the laser beam is made incident on the clad 26 as well and guided.

The present modification is to form the second light radiating unit 27b using such a clad 26.

In other words, for example, as shown in FIGS. 7 and 8, the first light radiating unit 27a is formed by adding, to a part of the core 25 projected from the clad 26 to the distal end side, a diffusing agent for irregularly reflecting light. For example, the second light radiating unit 27b is formed by adding, to a part of the distal end side of the clad 26, the diffusing agent for irregularly scattering light.

By forming the first and second light radiating units 27a and 27b using the core 25 and the clad 26 having different light amounts of the guided therapeutic light in this way, it is possible to differentiate the intensities of the therapeutic light radiated by the first and second light radiating units 27a and 27b.

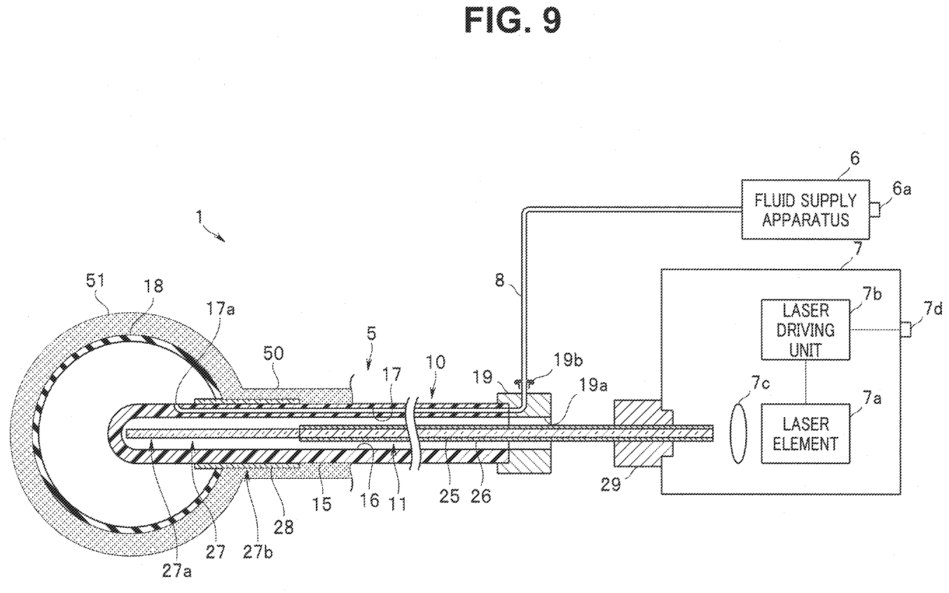

For example, it is also possible to form the second light radiating unit 27b by, as shown in FIG. 9, providing, in an outer circumference of the catheter main body 15, the light attenuating unit 28 provided in an outer circumference of the light radiating unit 27.

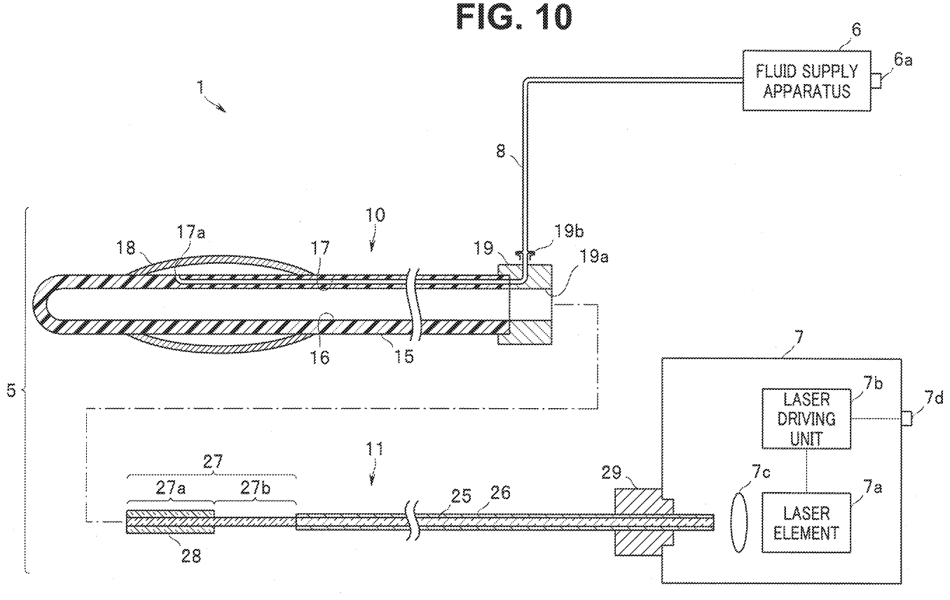

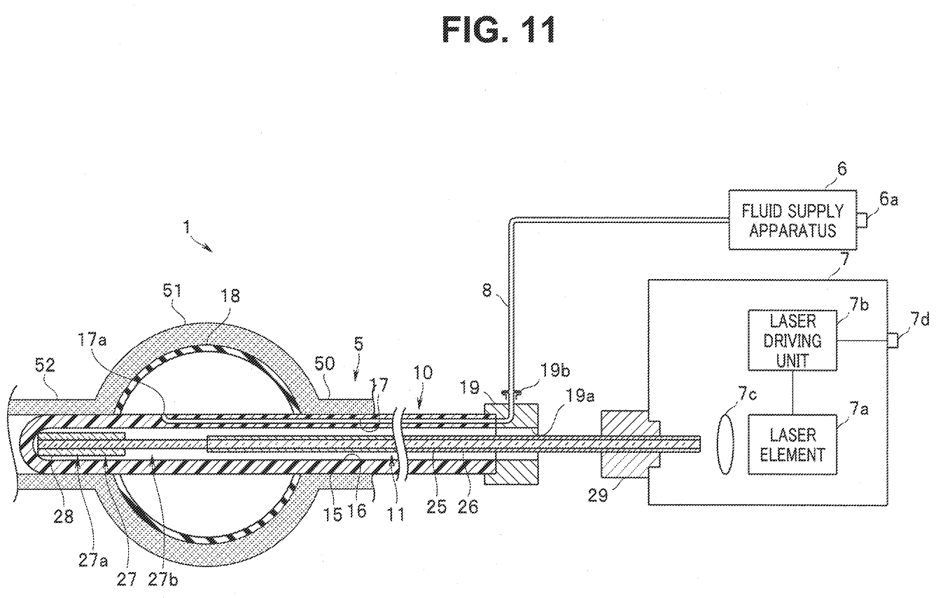

For example, as shown in FIGS. 10 and 11, it is also possible to use the light irradiation treatment instrument 5 in the present embodiment for treatment of the bladder 51 and a urinary tract 52 instead of the treatment of the urethra 50 and the bladder 51.

In this case, the balloon 18 is formed in a tubular shape opened at both ends. A distal end side opened end portion and a proximal end side opened end portion of the balloon 18 are liquid-tightly fixed to the outer circumferential surface of the catheter main body 15 by not-shown bobbin bonding or the like in front of and behind the distal end opening section 17a of the second conduit 17.

A sealed space communicating with the distal end opening section 17a of the second conduit 17 is formed halfway on the distal end side of the catheter main body 15 by the balloon 18 fixed in this way.

In other words, in the catheter 10 in the present modification, a region on the distal end side of the catheter main body 15 is projected further to the distal end side than the balloon 18 by a predetermined amount.

Consequently, for example, as shown in FIG. 11, in the catheter 10, it is possible to insert a partial region on the distal end side of the catheter main body 15 into the inside of the urinary tract 52 piercing through the bladder 51.

The light attenuating unit 28 is provided on the distal end side of the light radiating unit 27 in order to further attenuate intensity of therapeutic light radiated in the first light radiating unit 27a set in a region on the distal end side of the light radiating unit 27 than intensity of therapeutic light radiated in the second light radiating unit 27b set in a region on the proximal end side of the light radiating unit 27.

In the light irradiation treatment instrument 5 configured in this way, the catheter 10 is inserted through insides of the urethra 50, the bladder 51, and the urinary tract 52. The balloon 18 is expanded to the predetermined volume on the inside of the bladder 51, whereby the catheter 10 is positioned with respect to the bladder 51 and the urinary tract 52.

The optical fiber 11 is inserted into the first conduit 16 of the catheter 10 positioned in this way and is positioned with respect to the catheter 10, whereby the first light radiating unit 27a and the second light radiating unit 27b are positioned with respect to the urinary tract 52 and the bladder 51.

Therapeutic light is radiated from the first and second light radiating units 27a and 27b, whereby respective parts of surfaces (inner wall surfaces) of the urinary tract 52 and the bladder 51 are irradiated with illumination light having uniform intensity.

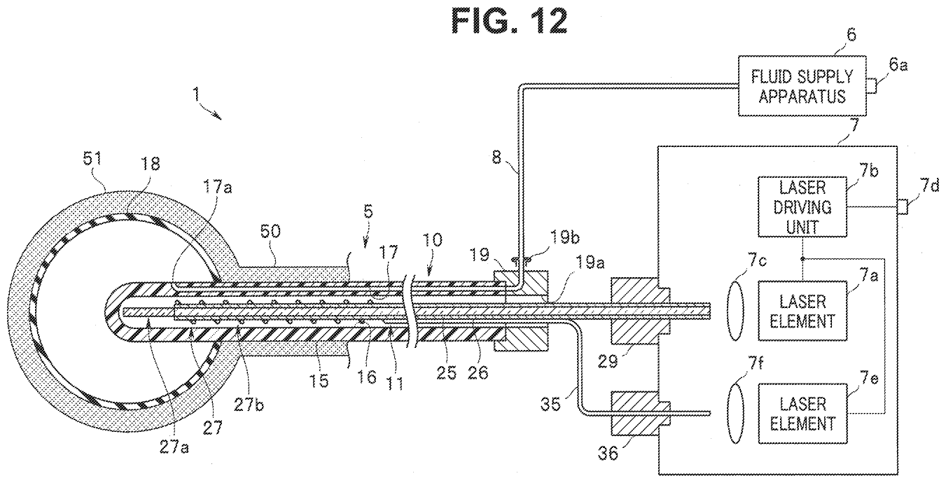

FIG. 12 relates to a second embodiment of the present invention. FIG. 12 is an explanatory diagram of a case in which light irradiation treatment into a bladder is performed using the optical treatment system. Note that the present embodiment is mainly different from the first embodiment explained above in that light sources in two systems are used as a light source for supplying therapeutic light to the first and second light radiating units 27a and 27b. Otherwise, the same components as the components in the first embodiment explained above are denoted by the same reference numerals and signs and explanation about the components is omitted as appropriate.

On the distal end side of the optical fiber 11 in the present embodiment, a part of the core 25 is projected from the clad 26. A projected region of the core 25 is set as the first light radiating unit 27a by adding a diffusing agent to the region.

In a region of the second light radiating unit 27b that irradiates a urethra, a second optical fiber 35 formed in a smaller diameter than the optical fiber 11 is wound on the outer circumference of the optical fiber 11. Since the total reflection condition is broken because the fiber is bent by the winding, light leaks to an outside without being propagated in this region. The second light radiating unit 27b is formed using this.

The proximal end side of the second optical fiber 35 is connected to the light source apparatus 7 via an optical connector 36. In other words, the proximal end side of the second optical fiber 35 is fixed to the optical connector 36 in a state in which the proximal end of the second optical fiber 35 is pierced through the optical connector 36. Consequently, when the optical connector 36 is connected to the light source apparatus 7, the optical connector 36 is capable of positioning a proximal end of the second optical fiber 35 in a predetermined position on the inside of the light source apparatus 7.

A second laser element 7e is provided on the inside of the light source apparatus 7. The second laser element 7e is disposed in a position opposed to, via a lens 7f, the proximal end of the second optical fiber 35 positioned on the inside of the light source apparatus 7. Consequently, the second laser element 7e is capable of making a laser beam functioning as therapeutic light incident on the second optical fiber 35.

The second laser element 7e is controlled to be driven by the laser driving unit 7b in synchronization with the laser element 7a. For example, an output of the first laser element 7a and an output of the second laser element 7e are adjusted such that intensity of light per unit area received by the bladder 51 or the like from the first light radiating unit 27a and intensity of light per unit area received by the urethra 50 or the like from the second light radiating unit 27b are equal.

Consequently, the second light radiating unit 27b is capable of radiating the therapeutic light in the same radiation time simultaneously with the first light radiating unit 27a.

According to such an embodiment, a system of a light source for supplying the therapeutic light to the first light radiating unit 27a and a system of a light source for supplying the therapeutic light to the second light radiating unit 27b are formed as separate systems. Consequently, it is possible to more precisely control the intensities of the therapeutic light radiated from the first and second light radiating units 27a and 27b.

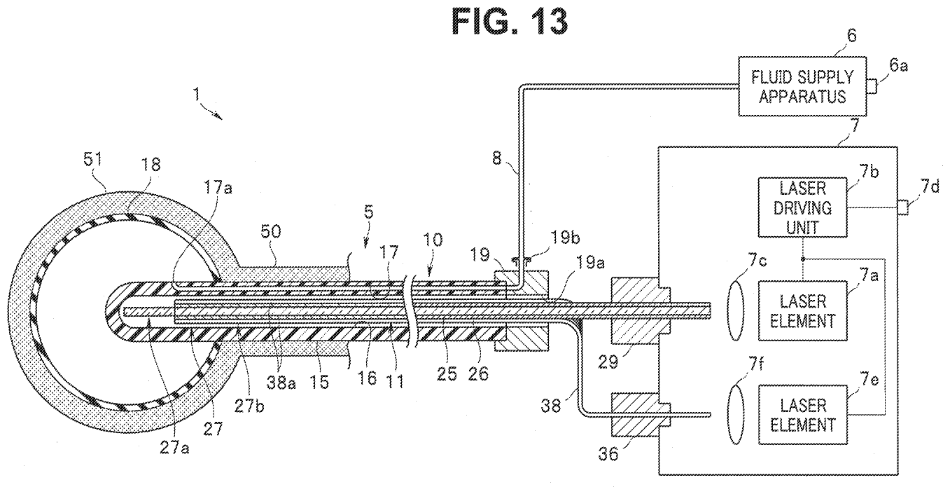

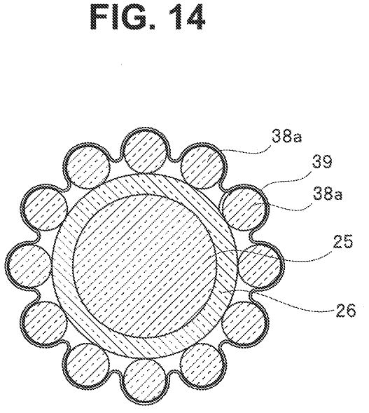

For example, as shown in FIGS. 13 and 14, it is also possible to adopt, instead of the second optical fiber 35, a second optical fiber 38, the distal end side of which is divided into a plurality of fiber sections, and annularly dispose divided optical fiber sections 38a around the optical fiber 11.

In this case, for example, as shown in FIG. 14, it is possible to fix the respective optical fiber sections 38a around the optical fiber 11 with a heat shrinkable tube 39 or the like having translucency.

The respective optical fiber sections 38a guide light to the second light radiating unit 27b. The guided light is used as light for performing treatment of the urinary tract 52 in the second light radiating unit 27b. The respective optical fiber sections 38a may not irradiate only the second light radiating unit 27b and may radiate an entire region from the proximal end side to the distal end side. The respective optical fiber sections 38a may be fixed in parallel around the optical fiber 11 as shown in FIG. 13 or may be wound in a spiral shape around the optical fiber 11.

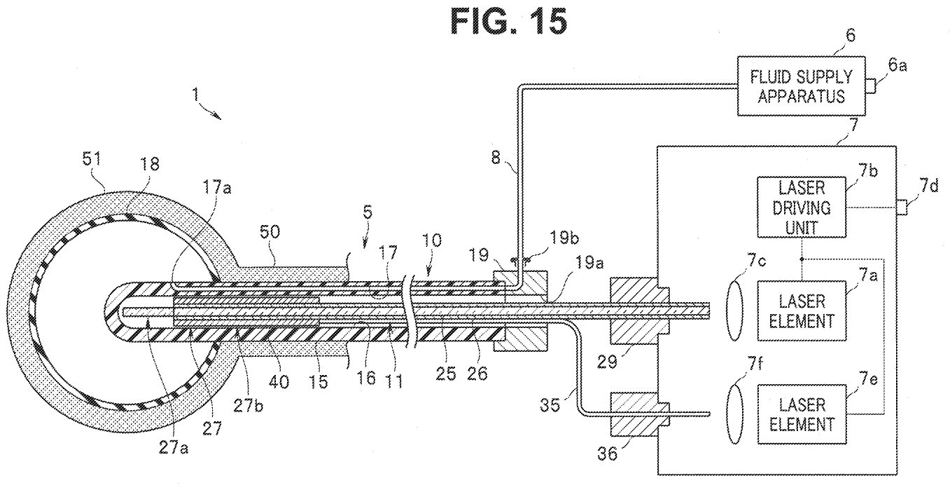

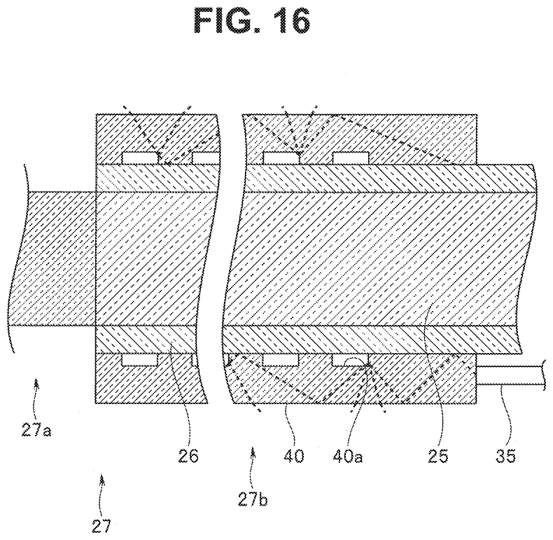

For example, as shown in FIGS. 15 and 16, it is also possible to provide a light guide plate 40 formed in a tube shape at the distal end of the second optical fiber 35 and configure the second light radiating unit 27b with the light guide plate 40.

In this case, for example, as shown in FIG. 16, a plurality of uneven sections 40a are provided on an inner circumferential surface of the light guide plate 40. By irregularly reflecting light in the uneven sections 40a, the light guide plate 40 is capable of radiating therapeutic light.

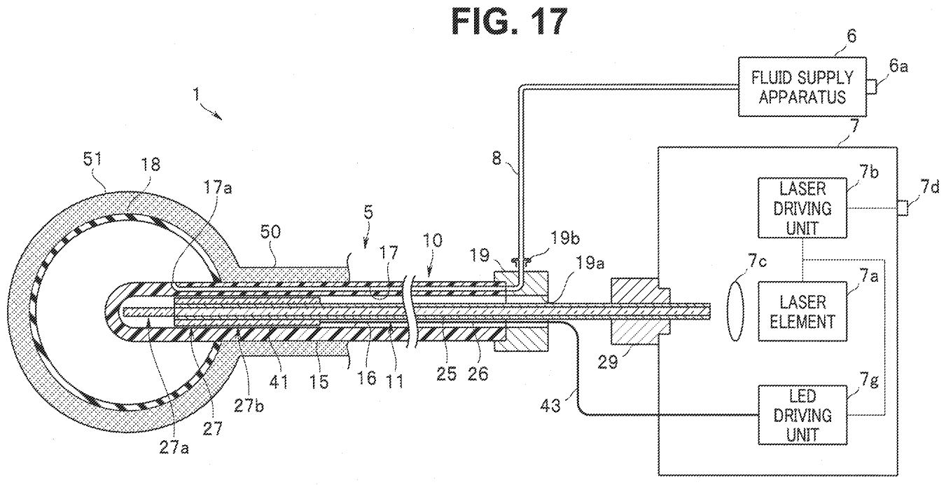

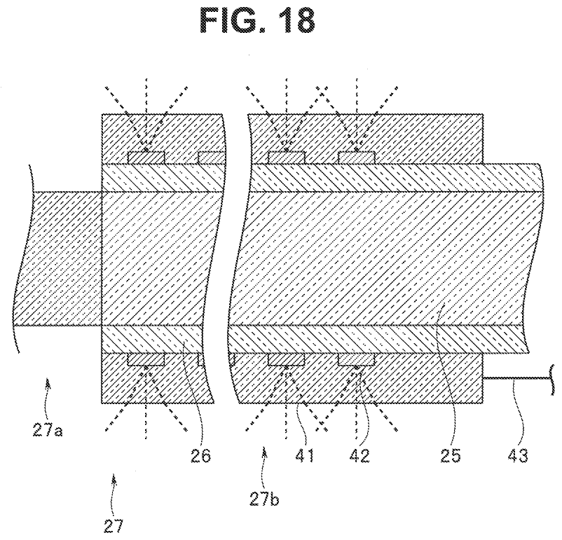

For example, as shown in FIGS. 17 and 18, it is also possible to configure the second light radiating unit 27b using an LED sheet 41 on which a plurality of LEDs 42, which are light emitting elements, are arrayed.

In this case, an LED driving circuit 7g for driving the LED sheet 41 is provided on the inside of the light source apparatus 7. The LED driving circuit 7g is electrically connected to the LED sheet 41 via a signal line 43.

For example, outputs of the respective LEDs 42 are set such that intensity of light per unit area received by the bladder 51 or the like from the first light radiating unit 27a and intensity of light per unit area received by the urethra 50 or the like from the second light radiating unit 27b are equal. Then, the LED driving circuit 7g is controlled by the laser driving unit 7b in synchronization with the laser element 7a. Consequently, the second light radiating unit 27b is capable of radiating therapeutic light in the same radiation time simultaneously with the first light radiating unit 27a.

In the embodiment explained above, an example is explained in which the laser driving unit 7b causes the laser element 7a and the second laser element 7e to emit light in synchronization. However, it is also possible to differentiate light emission times of the laser element 7a and the second laser element 7e.

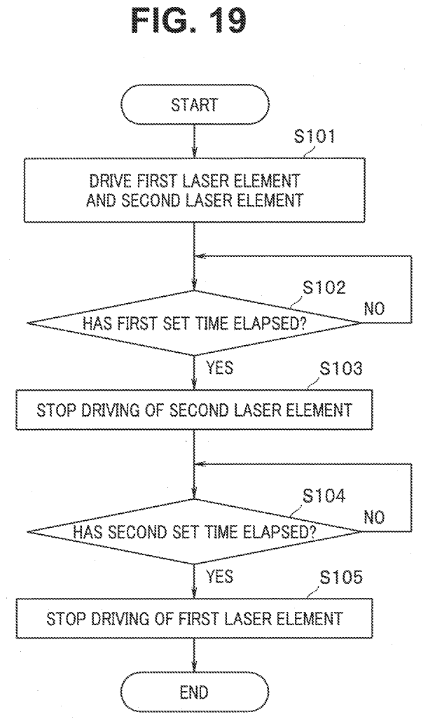

The laser driving unit 7b is capable of performing such control, for example, according to a flowchart of a radiation control routine for therapeutic light shown in FIG. 19.

This routine is executed by the laser driving unit 7b, for example, when the light source driving switch 7d is turned on. When the routine is started, first, in step S101, the laser driving unit 7b drives the laser element 7a and the second laser element 7e.

When proceeding from step S101 to step S102, the laser driving unit 7b checks whether a first setting time preset for the second laser element 7e has elapsed.

When determining in step S102 that the first setting time has not elapsed yet, the laser driving unit 7b stays on standby while maintaining a driving state of the laser element 7a and the second laser element 7e.

On the other hand, when determining in step S102 that the first setting time has elapsed, the laser driving unit 7b proceeds to step S103 and stops the driving of the second laser element 7e while maintaining the driving of the laser element 7a. Thereafter, the laser driving unit 7b proceeds to step S104.

When proceeding from step S103 to step S104, the laser driving unit 7b checks whether a preset second setting time has elapsed.

When determining in step S104 that the second setting time has not elapsed yet, the laser driving unit 7b stays on standby while maintaining the driving state of the laser element 7a.

On the other hand, when determining in step S104 that the second setting time has elapsed, the laser driving unit 7b proceeds to step S105, stops the driving of the laser element 7a, and, thereafter, leaves the routine.

By performing such control, it is possible to more precisely control optical energy of therapeutic light with which the respective parts are irradiated from the first and second light radiating units 27a and 27b.

The laser driving unit 7b may be realized by a computer including one or a plurality of processors, a logic circuit, a memory, an input and output interface, and a computer-readable recording medium. In that case, a program for realizing functions of respective components or an entire main body unit may be recorded in a recording medium. The functions may be realized by causing a computer system to read the recorded program and executing the program. For example, the processor is at least one of a CPU (central processing unit), a DSP (digital signal processor), or a GPU (graphics processing unit). For example, the logic circuit is at least one of an ASIC (application specific integrated circuit) or an FPGA (field-programmable gate array).

Note that the present invention is not limited to the respective embodiments explained above. Various modifications and changes are possible. The various modifications and changes are also within the technical scope of the present invention. For example, it goes without saying that the configurations of the respective embodiments and the respective modifications may be combined as appropriate.

It goes without saying that the light irradiation treatment instrument according to the present invention is also applicable to other parts in the body cavity.

What is claimed is:

1. A light irradiation treatment instrument comprising:
an insertion member insertable into an inside of a living body;
a positioning unit disposed between a first end and a second end of the insertion member and capable of positioning the insertion member with respect to the living body; and
a light radiating unit disposed between the first end and the second end and configured to radiate therapeutic light toward an outer side, intensity of the therapeutic light being different between a side closer to the first end than the positioning unit and a side closer to the second end than the positioning unit.

2. The light irradiation treatment instrument according to claim 1, further comprising a balloon fixed to the insertion member by a fixing unit, wherein
the positioning unit is the fixing unit for the balloon.

3. The light irradiation treatment instrument according to claim 1, wherein
the insertion member is inserted into a conduit and an inside of a hollow organ communicating with the conduit, and
the positioning unit comes into contact with a connecting portion of the conduit and the hollow organ to position the insertion member with respect to the conduit and the hollow organ.

4. The light irradiation treatment instrument according to claim 1, wherein the insertion member includes a light attenuating unit configured to attenuate the therapeutic light radiated from the light radiating unit.

5. A light irradiation treatment instrument comprising:
a catheter main body having translucency and being insertable into a conduit and an inside of a hollow organ communicating with the conduit;
a balloon having translucency and being provided in the catheter main body and configured to expand to thereby position the catheter main body with respect to the conduit and the hollow organ; and
an optical fiber having a predetermined length in a longitudinal direction, a first light radiating unit capable of radiating therapeutic light having first intensity and a second light radiating unit connected consecutively to the first light radiating unit in the longitudinal direction and capable of radiating the therapeutic light having second intensity different from the first intensity being formed on a distal end side, the optical fiber guiding the therapeutic light to the first light radiating unit and the second light radiating unit, wherein
when the optical fiber is inserted, the catheter main body positioned by the balloon positions one of the first light radiating unit and the second light radiating unit on an inside of the hollow organ and positions another one of the first light radiating unit and the second light radiating unit on an inside of the conduit and a joint portion of the conduit and the hollow organ.

6. The light irradiation treatment instrument according to claim 5, wherein the second intensity is lower than the first intensity.

7. The light irradiation treatment instrument according to claim 5, wherein the second intensity is higher than the first intensity.

8. The light irradiation treatment instrument according to claim 5, wherein the balloon includes a liquid holding unit configured to hold light-transmissible liquid around the first light radiating unit.

9. The light irradiation treatment instrument according to claim 8, wherein the balloon is inflated by the liquid.

10. The light irradiation treatment instrument according to claim 5, wherein
an outer circumferential surface of the first light radiating unit is formed by a conical surface having a first inclination angle, and
an outer circumferential surface of the second light radiating unit is formed by a conical surface having a second inclination angle different from the first inclination angle.

11. The light irradiation treatment instrument according to claim 5, wherein
the optical fiber includes a core and a clad covering an outer circumference of the core,
the second light radiating unit is a part of the clad covering the core, and
the first light radiating unit is a part of the core projected to the distal end side from the clad.

12. The light irradiation treatment instrument according to claim 5, wherein
the optical fiber includes a core and a clad covering an outer circumference of the core,
the first light radiating unit is a part of the core projected to the distal end side from the clad, and
the second light radiating unit is a part of the clad covering the core and a part of the clad on which a second optical fiber is wound.

13. The light irradiation treatment instrument according to claim 12, wherein
light from a first light source is guided to the optical fiber, and
light from a second light source different from the first light source is guided to the second optical fiber

14. An operation method for a light irradiation treatment instrument comprising:
inserting a catheter main body into a conduit and an inside of a hollow organ communicating with the conduit;
positioning the catheter main body with respect to the conduit and the hollow organ;
inserting an optical fiber into an inside of the catheter main body, the optical fiber having a predetermined length in a longitudinal direction, a first light radiating unit capable of radiating therapeutic light having first intensity and a second light radiating unit connected consecutively to the first light radiating unit in the longitudinal direction and capable of radiating therapeutic light having second intensity different from the first intensity being formed on a distal end side; and
with respect to the catheter main body, positioning one of the first light radiating unit and the second light radiating unit on an inside of the hollow organ and positioning another one of the first light radiating unit and the second light radiating unit on an inside of the conduit and a joint portion of the conduit and the hollow organ.

15. The operation method for the light irradiation treatment instrument according to claim 14, further comprising:
after the positioning of the first light radiating unit and the second light radiating unit with respect to the catheter main body, guiding light from a first light source to the first light radiating unit and guiding light from a second light source to the second light radiating unit;
ending the light guide to the second light radiating unit after a first setting time; and
ending the light guide to the first light radiating unit after a second setting time longer than the first setting time.
